(19) United States
(12) Patent Application Publication
(10) Pub. No.: US 2010/0268053 A1
(43) Pub. Date: Oct. 21, 2010

(54) Method and Device for Detecting USB Cable Connection

(75) Inventors: Alexander G. Ghesquiere, San Francisco, CA (US); Matthew Simmons, Pleasanton, CA (US); Christopher Ammon Myles, Alameda, CA (US)

Correspondence Address: JACKSON & CO., LLP, 6114 LA SALLE AVENUE, #507, OAKLAND, CA, 94611-2802, US

(73) Assignee: Abbott Diabetes Care Inc., Alameda, CA (US)

(21) Appl. No.: 12/761,374

(22) Filed: Apr. 15, 2010

Related U.S. Application Data
(60) Provisional application No. 61/169,691, filed on Apr. 15, 2009.

(51) Int. Cl. A61B 5/145; G06F 13/38; H01R 13/648

(52) U.S. Cl. 600/365; 710/100; 439/607.01; 600/309

(57) ABSTRACT

Methods, devices, and system including detecting the presence of an electrical connection in a data port of a medical device, the presence of the electrical connection associated with a variation in a signal level resulting from the electrical connection in the data port, and generating a control signal in response to the detected presence of the electrical connection in the data port, where generating the control signals includes one or more of outputting a notification associated with the presence of the electrical connection in the data port or modifying one or more operational parameters associated with the medical device are provided.

### RELATED APPLICATION

The present application claims priority under 35 U.S.C. §119(e) to U.S. provisional application No. 61/169,691 filed Apr. 15, 2009, entitled “Method and Device for Detecting USB Cable Connection”, the disclosure of which is incorporated herein by reference for all purposes.

### BACKGROUND

It is often desirable or necessary for medical devices, such as analyte measurement devices (e.g., in vitro blood glucose meters) to be in data communication with a peripheral device, such as a personal computer terminal for data communication or transfer. Data communication between the medical device and the personal computer include wired or wireless communication. A common form of wired communication includes the use of universal serial bus (USB) connection cables and interfaces, including for example, USB-A, USB-B, mini-USB-A, mini-USB-B, micro-USB-A, micro-USB-B, and USB On-The-Go mini and micro USB-A and USB-B cables and/or interfaces. Generally, USB interfaces are configured to provide power to low-consumption peripheral devices.

Certain electronic devices, including certain medical devices, are required to comply with electrical isolation requirements set forth in IEC-60601 providing medical electrical equipment safety standards, which, for example, require electrical isolation of the medical device circuits from, for example, a power supply source. One known technique for electrical isolation is by using opto-isolators. However, opto-isolators are often expensive to implement into a device and thus increase the cost associated with the manufacturing of the medical device.

### SUMMARY

One aspect of the present disclosure includes detecting the presence of an electrical connection in a data port of a medical device, the presence of the electrical connection associated with a variation in a signal level resulting from the electrical connection in the data port, and generating a control signal in response to the detected presence of the electrical connection in the data port, wherein generating the control signals includes one or more of outputting a notification associated with the presence of the electrical connection in the data port or modifying one or more operational parameters associated with the medical device.

In one embodiment, a cable port is provided which may comprise a cable port receptacle configured to receive a cable, a cable port receptacle shield provided within the cable port receptacle, where the cable port receptacle shield is coupled to a ground, and one or more contacts configured for detection of an insertion of a cable into the cable port receptacle.

These and other objects, features and advantages of the present disclosure will become more fully apparent from the following detailed description of the embodiments, the appended claims and the accompanying drawings.

### INCORPORATION BY REFERENCE

The following patents, applications and/or publications are incorporated herein by reference for all purposes: U.S. Pat. Nos. 4,545,382; 4,711,245; 5,262,035; 5,262,305; 5,264,104; 5,320,715; 5,356,786; 5,509,410; 5,543,326; 5,593,852; 5,601,435; 5,628,890; 5,820,551; 5,822,715; 5,899,855; 5,918,603; 6,071,391; 6,103,033; 6,120,676; 6,121,009; 6,134,461; 6,143,164; 6,144,837; 6,161,095; 6,175,752; 6,270,455; 6,284,478; 6,299,757; 6,338,790; 6,377,894; 6,461,496; 6,503,381; 6,514,460; 6,514,718; 6,540,891; 6,560,471; 6,579,690; 6,591,125; 6,592,745; 6,600,997; 6,605,200; 6,605,201; 6,616,819; 6,618,934; 6,650,471; 6,654,625; 6,676,816; 6,730,200; 6,736,957; 6,746,582; 6,749,740; 6,764,581; 6,773,671; 6,881,551; 6,893,545; 6,932,892; 6,932,894; 6,942,518; 7,041,468; 7,167,818; and 7,299,082; U.S. Published Application Nos. 2004/0186365; 2005/0182306; 2006/0025662; 2006/0091006; 2007/0056858; 2007/0068807; 2007/0095661; 2007/0108048; 2007/0199818; 2007/0227911; 2007/0233013; 2008/0066305; 2008/0081977; 2008/0102441; 2008/0148873; 2008/0161666; 2008/0267823; and 2009/0054748; U.S. patent application Ser. Nos. 11/461,725; 12/131,012; 12/242,823; 12/363,712; 12/495,709; 12/698,124; 12/699,653; 12/699,844; and 12/714,439 and U.S. Provisional Application Ser. Nos. 61/230,686 and 61/227,967.

### BRIEF DESCRIPTION OF DRAWINGS

FIG. 1 illustrates an analyte monitoring system in one embodiment of the present disclosure;

FIG. 2 is a block diagram of the analyte measurement device of FIG. 1 in one embodiment of the present disclosure;

FIG. 3 illustrates a cable detect module for detecting the insertion of a USB cable into a USB cable connection receptacle port in one embodiment of the present disclosure;

FIG. 4 illustrates a cable detect module for detecting the insertion of a USB cable into a USB cable connection receptacle port in one embodiment of the present disclosure;

FIGS. 5A and 5B illustrate a cable detect module for detecting the insertion of a USB cable into a USB cable connection receptacle port in one embodiment of the present disclosure;

FIGS. 6A and 6B illustrate a side view and perspective view, respectively, of one embodiment of the cable detect module of FIGS. 4A and 4B;

FIGS. 7A and 7B illustrate, respectively, a front view and bottom perspective view of a USB connection port including a cable detect module for detecting the insertion of a USB cable into the USB connection port in one embodiment;

FIGS. 8A-8C illustrate components of a USB communication port including a cable detect module in one embodiment;

FIGS. 9A-9E illustrate a top perspective view, a front (cable insertion direction) view, a bottom-front perspective view, a top-front perspective view and a bottom-rear perspective view, respectively, of a USB communication port including a cable detect module in one embodiment of the present disclosure;

FIG. 10 illustrates a cable detect module for detecting the presence or insertion of a USB cable into a USB cable connection receptacle port in one embodiment of the present disclosure;

FIG. 11 is a flow chart illustrating a cable insertion detection routine of a cable detect module in one embodiment; and

FIG. 12 is a flow chart illustrating an electrical isolation routine of a medical device in accordance with one aspect of the present disclosure.

### DETAILED DESCRIPTION

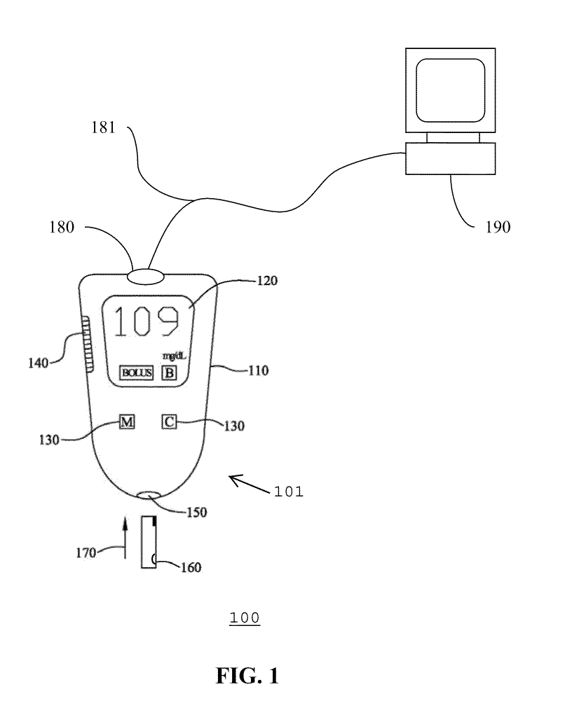

FIG. 1 illustrates an analyte monitoring system in one embodiment of the present disclosure. Referring to FIG. 1, an analyte monitoring system 100, in one embodiment, includes an analyte measurement device 101, such as an in vitro blood glucose meter or a data processing unit or a receiver unit of a continuous glucose monitoring system including an in vitro test strip port 150, coupled to a data processing and/or storage terminal 190, such as a computer, coupled by a communication cable 181. The analyte measurement device 101 may be a continuous, semi-continuous, or discrete analyte measurement device. Additional detailed descriptions of such measurement device and associated system are provided in U.S. Pat. Nos. 5,262,035; 5,264,104; 5,262,305; 5,320,715; 5,593,852; 6,175,752; 6,650,471; 6,746,582; 7,041,468, and in application Ser. Nos. 10/745,878 filed Dec. 26, 2003 entitled “Continuous Glucose Monitoring System and Methods of Use”, 12/699,653 filed Feb. 3, 2010 entitled “Multi-Function Analyte Test Device and Methods Therefor” and 12/699,844 filed Feb. 3, 2010 entitled “Multi-Function Analyte Test Device and Methods Therefor”, the disclosures of each of which are incorporated herein by reference for all purposes.

Referring to FIG. 1, in one embodiment, the analyte measurement device 101 includes a housing 110 with a display unit 120 provided thereon. Also shown in FIG. 1 is a plurality of input buttons 130, each configured to allow the user of the analyte measurement device 101 to input or enter data or relevant information associated with the operation of the analyte measurement device 101. For example, the user of the analyte measurement device 101 may operate the one or more input buttons 130 to enter a calibration code associated with a test strip 160, device setting information such as, for example, time and date information, for use in conjunction with the analyte measurement device 101.

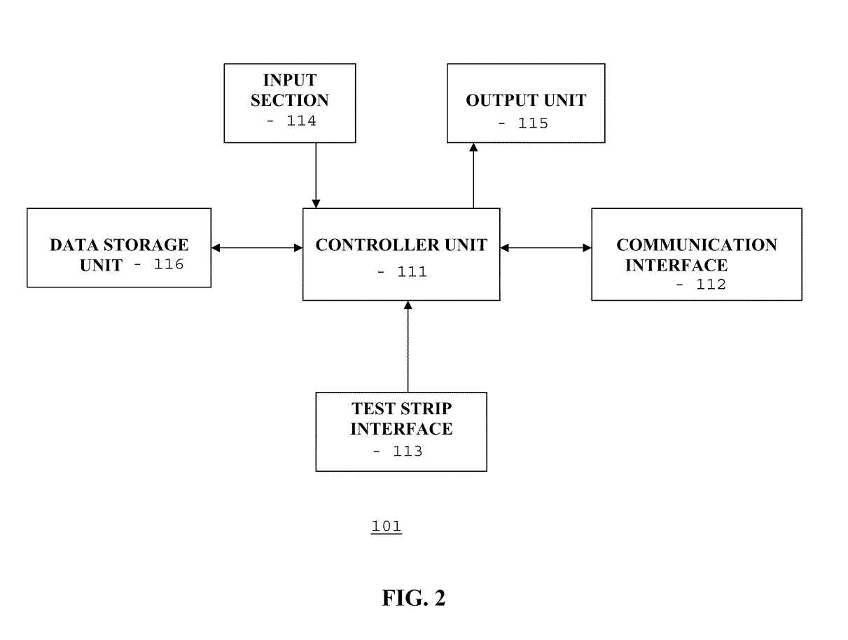

Referring back to FIG. 1, also shown is input unit 140 which, in one embodiment, may be configured as a jog dial, or the like, and provided on the housing 110 of the analyte measurement device 101. Also shown is a strip port 150 which is configured to receive the test strip 160 (with fluid sample provided thereon) substantially in the direction as shown by the directional arrow 170. In operation, when the test strip 160 with the fluid sample, such as a blood sample, is inserted into the strip port 150 of the analyte measurement device 101, a control unit 111 (FIG. 2), such as a microprocessor or an application specific integrated circuit (ASIC), of the analyte measurement device 101 may be configured to determine the analyte level in the fluid sample (such as blood sample), and display the determined analyte level on the display unit 120.

FIG. 2 is a block diagram of the analyte measurement device 101 of FIG. 1 in one embodiment of the present disclosure. Referring to FIG. 2, the analyte measurement device 101 includes a controller unit 111 operatively coupled to a communication interface 112 and configured for bidirectional communication. The controller unit 111 is further operatively coupled to a test strip interface 113, an input section 114 (which, for example, may include the input unit 140 and the plurality of input buttons 130 as shown in FIG. 1), an output unit 115, and a data storage unit 116.

Referring still to FIG. 2, in one embodiment of the present disclosure, the test strip interface 113 is configured for signal communication with the inserted test strip 160 (FIG. 1) for determination of the analyte level of the fluid sample on the test strip 160. In addition, the test strip interface 113 may include an illumination segment which may be configured to illuminate the strip port 150 (FIG. 1) using a light source such as, for example, but not limited to, a light emitting diode (LED), for example, during the test strip 160 insertion process to assist the user in properly and accurately inserting the test strip 160 into the strip port 150. Details of example configurations of such devices are provided in application Ser. No. 10/861,626, the disclosure of which is incorporated herein by reference for all purposes.

In a further aspect of the present disclosure, the test strip interface 113 may be configured with a physical latch or securement mechanism internally provided within the housing 110 of the analyte measurement device 101 (FIG. 1) such that when the test strip 160 is inserted into the strip port 150, the test strip 160 is retained in the received position within the strip port 150 until the sample analysis is completed. Examples of such physical latch or securement mechanism may include a uni-directionally biased anchor mechanism, or a pressure application mechanism to retain the test strip 160 in place by applying pressure on one or more surfaces of the test strip 160 within the strip port 150.

Referring back to FIG. 2, the output unit 115 may be configured to output display data or information including the determined analyte level on the display unit 120 (FIG. 1) of the analyte measurement device 101. In still a further aspect of the present disclosure, the output unit 115 and the input section 114 may be integrated, for example, where the display unit 120 is configured as a touch sensitive display where the patient may enter information or commands via the display area using, for example, a stylus or any other suitable input device, and where, the touch sensitive display is configured as the user interface in an icon driven environment.

Referring yet again to FIG. 2, the communication interface 112 in one embodiment of the present disclosure includes a USB port configured for communication with, for example, a data processing and/or storage terminal 190 (FIG. 1), such as a computer, via a communication cable 181. In other embodiments, the communication interface 112 may also include other wired or wireless communication interfaces, including, for example, bi-directional radio frequency (RF) communication with other devices to transmit and/or receive data to and from the analyte measurement device 101.

Referring back to FIG. 1, in one embodiment, the analyte measurement device 101 further includes a communication port 180, such as a USB communication port, housed therein or coupled thereto for connection to the data processing and/or storage terminal 190 via the communication cable 181. Communication between the analyte measurement device 101 and the data processing and/or storage terminal 190 via the communication cable 181 may include transfer of measured, processed, and/or stored data between the analyte measurement device 101 and the data processing and/or storage terminal 190, such as, for example, logged historical data of measured or determined blood glucose level of the user with associated time and/or date stamp indicating the time and/or date of measurement.

In one aspect, the data processing and/or storage terminal 190 may be configured for further data processing, storage, and/or analysis of the data received from the analyte measurement device and further may be configured for further transmission to, for example, a server database or a treating professional. Moreover, the data processing and/or storage terminal 190 may also be configured to transmit, for example, instruction or calibration information, to the analyte measurement device 101.

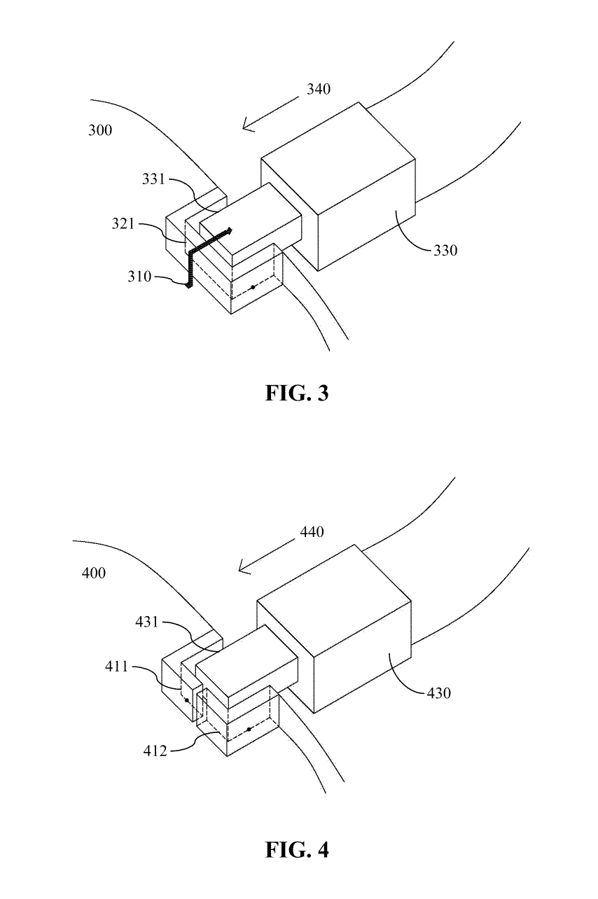

Referring again to FIG. 1, the USB communication port 180 may also include a module for detection of the presence or insertion of a USB cable 181 into the communication port 180, as described in further detail below and in conjunction with FIGS. 3-10. The detection of the presence or insertion of the USB cable 181 into the communication port 180 may be used for, among others, a signal to the analyte measurement device 101 to disable any analyte testing features during the duration of time when the USB cable 181 is inserted into the communication port 180.

FIG. 3 illustrates a cable detect module for detecting the insertion of a USB cable into a USB cable connection receptacle port in one embodiment of the present disclosure. The USB connection for an analyte monitoring device is used herein for exemplary purposes and is not to be construed as limiting to particular embodiments, and it is to be understood that the methods, components, modules, and systems described herein may be implemented with a variety of connection types and/or protocols and/or device hardware.

Referring to FIG. 3, in one embodiment, the cable detect module of a medical device may include a single conductive contact 310. More specifically, the contact 310 may include a conductive material, such as, but not limited to, gold, silver, copper, aluminum, and the like positioned at a USB receptacle port 321 located within the housing 300 of the medical device. In one embodiment, the initial position of the contact 310 provides for an incomplete or open electrical connection. The contact 310 may be positioned such that that when a USB cable 330 is inserted into the USB receptacle port substantially in the direction shown by directional arrow 340, the insertion movement biases the contact 310 to contact the shield 331 of the USB cable 330 and the shield of the USB receptacle port 321. In one aspect, the shield of the USB receptacle port 321 is connected to ground of the medical device, and thus, when a USB cable 330 is inserted into the USB receptacle port to electrically couple the contact 310 of the detect module to the USB receptacle port shield 321, the contact 310 of the detect module is also connected to ground.

In one embodiment, the change in position of the contact 310 resulting from the insertion of the USB cable 330 into the USB receptacle port, completes the electrical connection. In one aspect, the contact 310 and the electrical connection is monitored by the control unit 111 (FIG. 2) of the medical device. Accordingly, the control unit 111 may be configured to detect a signal level change when the electrical connection is closed resulting in a closed circuit. Alternatively, the change in position of the contact 310 due to the insertion of the USB cable 330 into the receptacle port, may result in, among others, a shorting of an electrical connection, a change in voltage of an electrical circuit loop, a change in resistance of an electrical circuit loop, or a change in current of an electrical circuit loop, each of which, in one aspect, may be monitored and detected by the control unit 111.

In one aspect, the control unit 111 is configured to adjust the functionality of the medical device by, for example, generating and outputting a notification to the user, or disabling one or more medical device operation functions when the control unit 111 detects that the contact 310 is connected to the ground terminal. In one embodiment, the control unit 111 may be configured to disable the analysis function of the medical device configured to analyze fluid sample provided on a test strip inserted into a strip port of the medical device.

Referring still to FIG. 3, in one aspect, the subsequent removal of the USB cable 330 from the USB receptacle port results in the contact 310 no longer contacting the shield of the USB receptacle port 321, and thus the contact 310 is no longer connected to ground. When it is detected by the control unit 111 (FIG. 2) that the contact 310 is no longer connected to ground, it is determined that the USB cable 330 is not inserted into the USB receptacle port, and thus the control unit 111 resumes normal operation.

FIG. 4 illustrates a cable detect module for detecting the presence or insertion of a USB cable into a USB cable connection receptacle port in one embodiment of the present disclosure. Referring to FIG. 4, the cable detect module of a medical device may include a pair of contacts 411, 412. The pair of contacts 411, 412, in one embodiment may be integrated into the shield of the receptacle port located within a housing 400 of the medical device. When a USB cable 430 is inserted into the receptacle port substantially in the direction shown by directional arrow 440, an electrical connection is established between the two contacts 411, 412. The electrical connection between the two contacts 411, 412, in one embodiment, results from the contact with the shield 431 or other conductive section of the USB cable 430.

In one aspect, the pair of contacts 411, 412 are monitored by a control unit 111 (FIG. 2), and when the electrical connection is detected by the control unit, the control unit may be configured to prompt the user of the medical device notifying of such connection, and/or adjust the functionality of the medical device, for example, by discontinuing or disabling one or more functions of the medical device. Furthermore, when the USB cable is subsequently removed from the receptacle port, the pair of contacts 411, 412 may be configured (for example, with sufficient bias, torsion or spring force) such that they are physically separated and no longer in electrical contact, and the control unit may determine that the USB cable is no longer present in the USB receptacle port or has been withdrawn from the port, and may resume normal operation of the medical device.

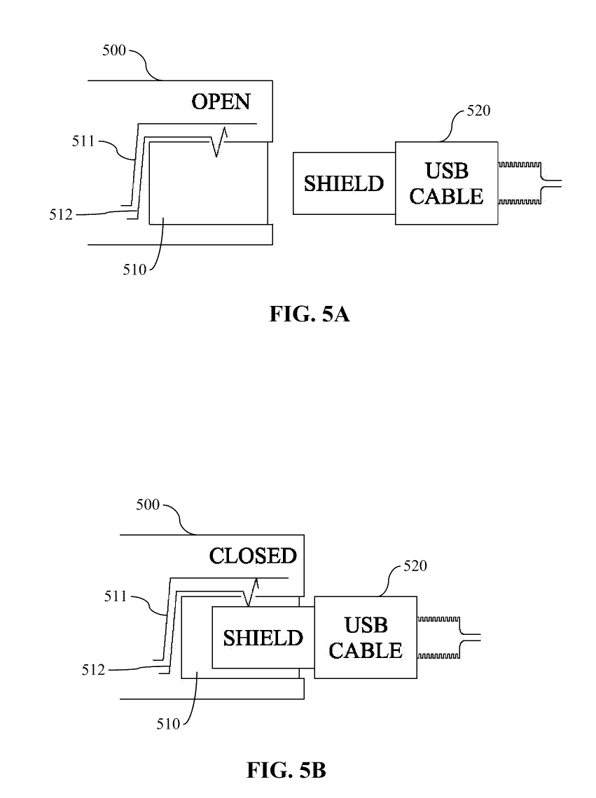

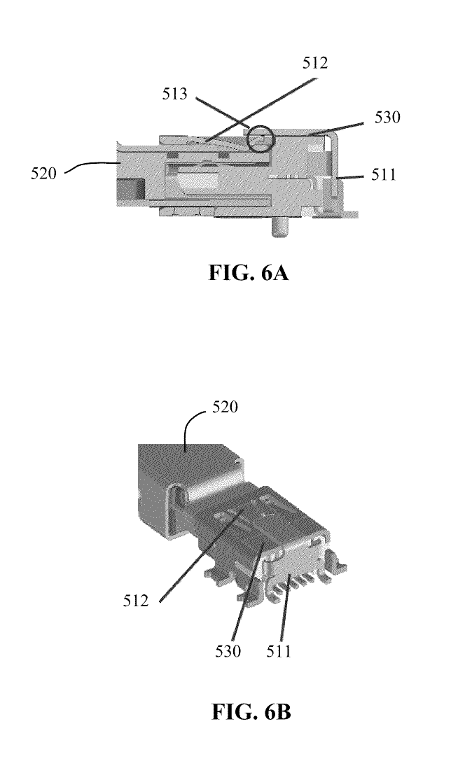

FIGS. 5A and 5B illustrate a cable detect module for detecting the presence or insertion of a USB cable into a USB cable connection receptacle port in one embodiment of the present disclosure. FIGS. 6A and 6B illustrate side and perspective views of the cable detect module of FIGS. 5A and 5B.

Referring to FIGS. 5A, 5B, 6A and 6B, in one embodiment, a USB connection port 510 of a medical device 500, includes two contacts 511, 512 that are separated when no USB cable 520 is present or inserted in the USB connection port 510. When a USB cable 520 is inserted into the USB connection port 510 the two contacts 511, 512 are forced to contact one another as shown in FIG. 5B and indicated by reference numeral 513 of FIG. 6A. The two contacts 511, 512 may be configured with bias or spring-like characteristics such that application of a predetermined force, for example, by the shield 521 or another component of the USB cable 520 in conjunction with the insertion movement of the cable 520 results in the two contacts 511, 512 to electrically couple. Upon contact between the two contacts 511, 512, an electrical connection is established. Alternatively, in other embodiments, the contact between the two contacts 511, 512 may result in a change in voltage, current, and/or resistance of on the electrical connection which may be detected by the control unit 111 (FIG. 2) or processing terminal of the medical device 500.

More specifically, the position of the two contacts 511, 512 and/or the resulting signal level changes due to the subsequent completed, shorted, or altered electrical connection by the movement of the two contacts 511, 512, may be monitored by a control unit 111 (FIG. 2) of the medical device 500. Upon detection by the control unit 111 of the signal level changes resulting from the movement of the contacts 511, 512 of the cable detect module, in one aspect, the control unit 111 may be configured to generate a signal or notification to indicate to the user that a USB cable 520 has been inserted into the USB connection port 510. Alternatively, as discussed above, the control unit 111 may be configured to disable or alter one or more functionalities of the medical device 500 when the signal level change is detected.

In one embodiment, the cable detection contacts 511, 512 may be configured to not directly contact the shield of the USB cable 520. For example, an insulating layer (not shown) may be provided between the shield of the USB cable 520 and contact 511. This insulation layer may also act as a barrier to prevent electrical shorts. Furthermore, an insulation layer 530 may also be provided between contact 512 and the USB connection port 510 to prevent electrical shorts.

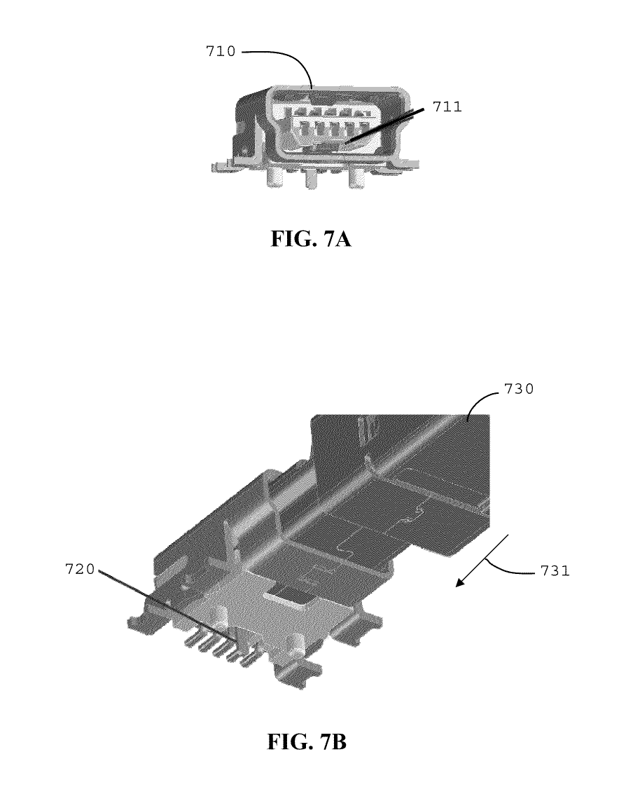

FIGS. 7A and 7B illustrate, respectively, a front view and bottom perspective view of a USB connection port including a cable detect module for detecting the presence or insertion of a USB cable into the USB connection port in one embodiment. Referring to FIGS. 7A and 7B, in one embodiment, a contact 711 is provided within the receptacle of a USB connection port 710. When a USB cable 730 is inserted into the USB connection port 710, substantially in the direction shown by directional arrow 731, the USB cable 730 contacts the contact 711. The contact 711 is coupled to a pin 720 provided through a hole, for example, of the connection port 710.

In one embodiment, the pin 720 is operatively coupled to a control unit 111 (FIG. 2). Upon contact between the USB cable 730 and contact 711, an electrical signal is routed through the pin 720 and detected by the control unit 111. The electrical signal routed through the pin 720 is indicative of whether or not a USB cable 730 is inserted into the USB connection port 710. Accordingly, upon detection by the control unit 111 of the presence or insertion of the USB cable 730 in the USB connection port 710, the control unit 111 may be configured to generate a notification to the user of the medical device and/or adjust the functionality of the medical device. In one embodiment, the control unit 111 is configured to disable all features of the medical device related to measurement of medical data, such as analysis of a fluid sample provided on a test strip and inserted into a test strip port of the medical device.

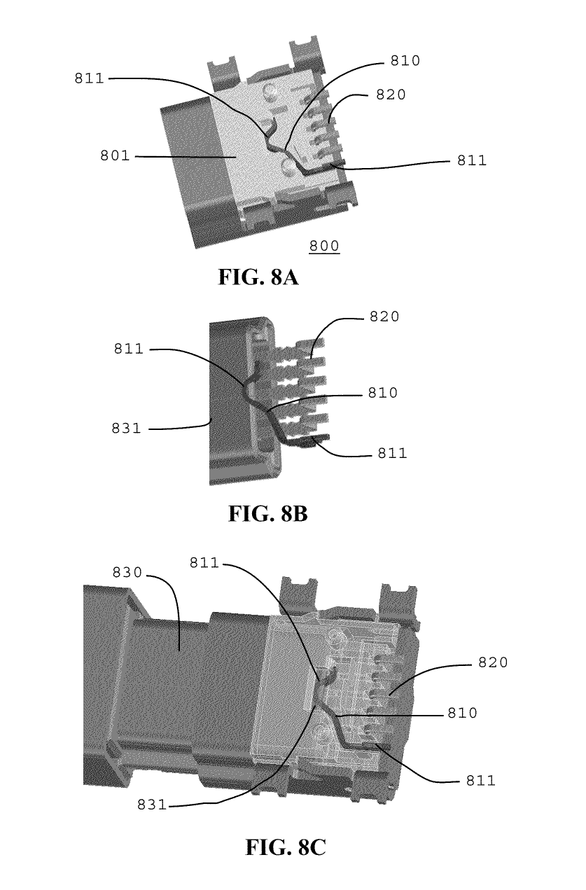

FIGS. 8A-8C illustrate components of a USB communication port including a cable detect module in one embodiment. In particular, FIG. 8A illustrates the USB communication port including the cable detect module without a USB cable inserted, FIG. 8B illustrates the pin connections of the USB communication port including the cable detect module upon insertion of a USB cable into the USB communication port, and FIG. 8C illustrates the USB communication port including the cable detect module with a USB cable inserted therein.

Referring to FIGS. 8A-8C, in one embodiment, a USB communication port 800 includes a cable detect module including a conductive contact 810 provided at the USB communication port 800. In one aspect, the conductive contact 810 may be separated from conductive portions of the USB communication port 800 by an insulating layer 801. A first portion 811 of the conductive contact 810 is provided through a hole or slot in the insulating layer 801, where upon insertion of a USB cable 830 into the USB communication port 800, a conductive portion 831 of the USB cable 830 establishes electrical contact with the first portion 811 of the conductive contact 810. In one embodiment, the conductive portion 831 of the USB cable 830 includes the USB cable shield.

Contact between the first portion 811 of the conductive contact 810 and the conductive portion 831 of the USB cable 830 in one embodiment results in a signal level change in the electrical connection associated with the conductive contact 810. In one aspect, the signal level change is monitored and detected by a control unit 111 (FIG. 2) in signal communication with the conductive contact 810 through, for example, a pin connection section 812 of the conductive contact 810. The pin connection section 812 of the conductive contact 810 may be configured to be one of a bank of pin connections 820 configured for bi-directional communication between the USB communication port 800 and the control unit 111.

In one embodiment, the USB communication port 800 maybe housed in or coupled to a medical device, such as an analyte measurement device (e.g., an in vitro blood glucose meter). Upon detection of the signal level change resulting from the electrical connection between the conductive contact 810 of the cable detect module and the USB cable 830, the control unit 111 in one aspect is configured to generate and output a notification to the user of the medical device and/or adjust or disable one or more operational parameters of the medical device. For example, as discussed above, in one embodiment, the control unit 111 may be configured to discontinue medical data measurement procedures and/or disable medical data measurement functions, such as analyzing the fluid sample provided on a test strip inserted into the strip port of the medical device (see, for example, FIG. 1).

Referring back to the Figures, when the USB cable 830 is subsequently removed or withdrawn from the USB communication port 800, the conductive contact 810 is no longer in electrical contact with the USB cable 830, and the signal level of the electrical connection associated with the conductive contact 810 returns to its initial level. Accordingly, in one aspect, the change in the signal level is detected by the control unit 111 and the control unit 111 determines the USB cable 830 is no longer connected to the USB communication port 800, and may configure the medical device to resume normal operation.

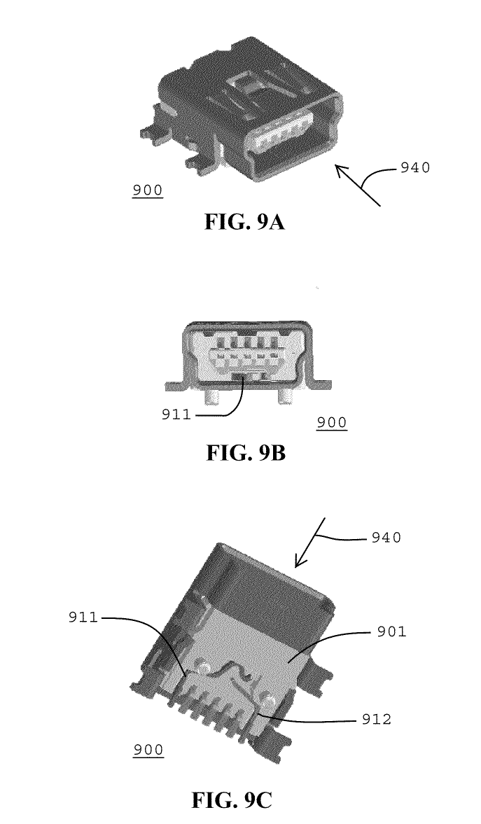

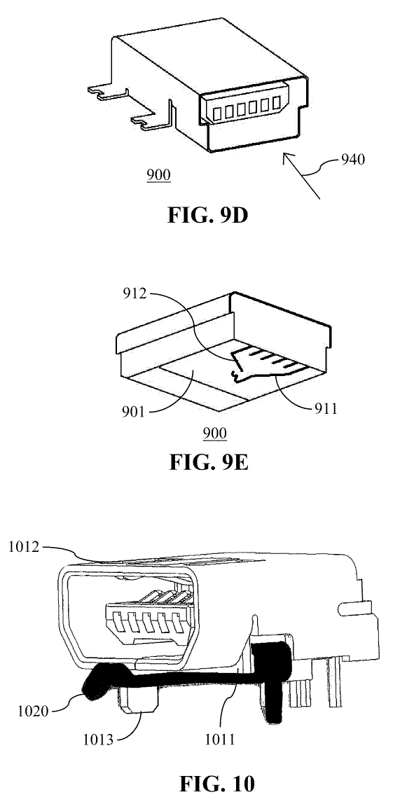

FIGS. 9A-9E illustrate a top perspective view, a front (cable insertion direction) view, a bottom-front perspective view, a top-front perspective view and a bottom-rear perspective view, respectively, of a USB communication port including a cable detect module in one embodiment of the present disclosure. Referring to FIGS. 9A-9E, a USB communication port 900 including a cable detect module is configured to accept insertion of a USB cable (not shown) substantially in the direction indicated by directional arrow 940.

Still referring to FIGS. 9A-9E, in one embodiment, the cable detect module includes one or more conductive contacts 911, 912 provided at the USB communication port 900. While FIGS. 9A-9E show the cable detect module with two conductive contacts, within the scope of the present disclosure, the cable detect module may include more than two conductive contacts. In one aspect, upon insertion of a USB cable (not shown) into the USB communication port 900, the insertion force of the USB cable results in the conductive contact 911 repositioning from an initial position to an inserted position such that when conductive contact 911 is in the inserted position, conductive contact 911 contacts conductive contact 912.

The contact between conductive contact 911 and conductive contact 912, in one embodiment, results in an electrical connection where a signal level change is detected by a control unit in signal communication with the electrical connection associated with conductive contacts 911 and 912. Upon detection of the signal level change, the control unit may determine on one embodiment that a USB cable is inserted into the USB communication port 900.

Furthermore, in one embodiment, when a USB cable is removed or withdrawn from the USB communication port, conductive contact 911 may be configured to return to its initial position from the inserted position. Conductive contact 911, in one aspect, is comprised of a conductive material with an elasticity coefficient or configured with sufficient spring or bias force, such that conductive contact 911 may be repositioned between the initial position and the inserted position without breaking or being permanently positioned in the inserted position. Upon returning to the initial position of conductive contact 911, the electrical connection associated with conductive contacts 911 and 912 is terminated and an open-circuit results. The change in the electrical connection from closed to open circuit results in a signal level change detected by the control unit. In turn, the control unit may be configured to determine that the USB cable is removed from the USB communication port 900.

In one embodiment, the conductive contacts 911, 912 may be separated from the conductive portions of the USB communication port 900 by an insulating layer 901. In another embodiment, a first portion of conductive contact 911 may be provided through a hole or slot in the insulating layer 901, such that upon insertion of a USB cable into the USB communication port 900, a portion of USB cable will directly or indirectly contact the first portion of conductive contact 911. In one embodiment, the portion of the USB cable that contacts conductive contact 911 may include the USB cable shield. In another embodiment, the portion of the USB cable that contacts conductive contact 911 includes a non-conductive portion of the USB cable. In still a further aspect, the first portion of conductive contact 911 may be protected by an insulating layer to prevent shorts and/or electrical contact with a conductive portion of the USB cable and/or a conductive portion of the USB communication port 900.

FIG. 10 illustrates a cable detect module for detecting the presence or insertion of a USB cable into a USB cable connection port in one embodiment of the present disclosure. Referring to FIG. 10, in one embodiment, a contact 1020 is provided that protrudes beyond the front of the connection port 1010, where the contact 1020 is configured to contact a USB cable upon insertion into the connection port 1010. In one embodiment, the contact 1020 may be embedded into or integrated with the body 1011 of the connection receptacle and isolated from the conductive shell 1012 of the connection port 1010. Furthermore, the contact 1020 may replace one or more of the mounting components 1013 of the USB connection port 1010.

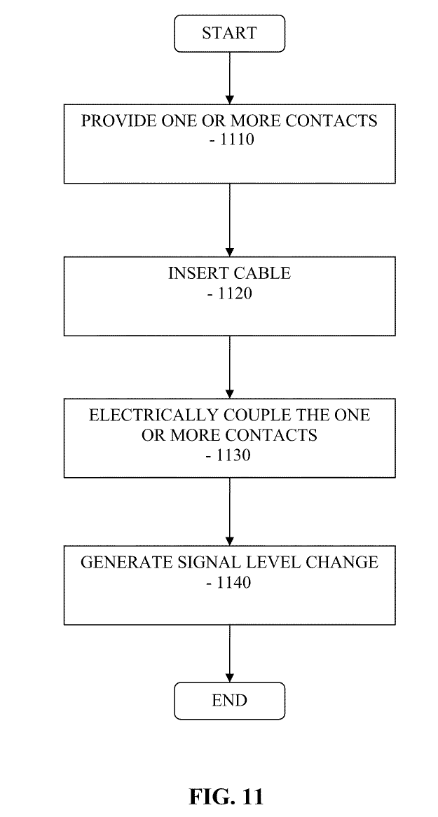

FIG. 11 is a flow chart illustrating a cable insertion detection routine of a cable detect module in one embodiment of the present disclosure. Referring to FIG. 11, in one embodiment, one or more contacts are provided (1110) in a cable detect module of a communication port, such as the cable detect modules described above and illustrated in FIGS. 3-10. Insertion of a cable (1120) into the communication port causes the one or more contacts to electrically couple (1130). The electric coupling of the one or more contacts causes the generation of a signal level change (1140), which is detected by a control unit in operational communication with the one or more contacts. The detection of the signal level change acts as an indication to the control unit that a cable is inserted into the communication port.

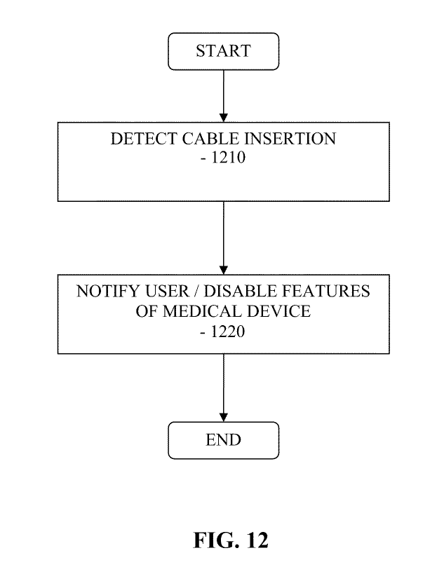

FIG. 12 is a flow chart illustrating electrical isolation routine of a medical device in one aspect of the present disclosure. A medical device such as, for example, an in vitro blood glucose meter may include a communication port, for example a USB connection port. Referring to FIG. 12, the medical device may include a cable detect module at the connection port, such that when a USB cable is inserted into the connection port, the insertion or presence of the USB cable is detected (1210).

The cable detect module in one embodiment may be monitored by a control unit of the medical device, and upon detection of the presence or insertion of the USB cable, the control unit is configured to notify the user to inform the user of the detected USB cable insertion or presence, and/or disable or modify one or more operational functions of the medical device (1220). For example, for an analyte monitoring device, the analyte measurement features, such as the analysis of a fluid sample provided on an analyte test strip inserted into a test strip port of the medical device, may be disabled.

In one embodiment, a cable connection port of a device, for example a USB connection port of an analyte monitoring device, includes a component configured for detection of the connection of a connection cable. Upon detection of a connected cable, a control unit may be configured to adjust or modify the one or more operational parameters of the medical device. Such operational parameters may include, but are not limited to, disabling medical measurement functions of the device, such as blood glucose level measurements of an analyte monitoring device.

The component of a cable connection port of a device for detection of the connection of a cable, in one embodiment, may include a contact where when a cable is inserted into the connection port, the contact is connected to a ground of the device. The contact may be comprised of a conductive material, such as, but not limited to, gold, silver, copper, aluminum, etc. In another embodiment, the component of a cable connection port of a device for detection of the connection of a cable is a pair of contacts. When a cable is inserted into the cable connection port, a metallic or other conductive surface, causes an electrical connection of the pair of contacts. The electrical connection of the contacts may be detected by a control unit indicating a presence of a cable in the port.

In a further aspect, the component of a cable connection port of a device for detection of the connection of a cable may include two (or more) contacts initially physically separated. Upon insertion of a cable into the connection port, the two (or more) contacts are forced into physical contact with one another, thus causing a signal level change of a circuit, which is an indication that a cable has been inserted into the connection port. The signal level change is detected by a control unit which may be configured to adjust the functionality of the device accordingly.

In one embodiment, the component of a cable connection port of a device for detection of the connection of a cable is a power detection module configured to detect a change in current, voltage, or power, thereby detecting the presence of a power signal along the cable. For example, in the case of USB, when a USB cable is plugged into both a first and second device, a power signal is generated and transmitted along the USB cable line. This power signal can be detected by the power detection module, and further a control unit in operational communication with the power detection module, may be configured to notify the user of the detected signal, and/or modify or adjust one or more operational parameters of the device.

In other embodiments, a USB cable may be provided that includes one or more components, for the detection of the insertion of the USB cable into a USB connection port receptacle. For example, the USB cable may include a contact or pin through which a signal indicating the insertion of the USB cable into the USB connection port may be routed.

As discussed above, embodiments of the present disclosure include methods and devices for detecting the connection of a USB cable in a receptacle port of a medical device. The detection of a USB cable connection, in one embodiment, is achieved by one or more conductive contacts configured to provide an indication associated with the presence or insertion a USB cable into a USB receptacle port. In one aspect, the presence or insertion of the USB cable into a corresponding USB interface or port, and subsequent direct or indirect contact with, or movement of, the conductive contacts, may be monitored by a control unit, such as a microprocessor of the medical device.

The control unit may be in signal communication with the conductive contacts and monitors an electrical signal level change generated as a result of the contact with, or movement of, the conductive contacts resulting from the presence or insertion of the USB cable into the corresponding USB interface or port. The signal level change may result from the contact with, or movement of, the conductive contacts which, in one embodiment, provides a change in measurable voltage, current, or resistance associated with the USB interface port detected by the control unit of the medical device, for example.

Upon the detection of the insertion of the USB cable by the control unit, in one aspect, the control unit may be configured to perform one or more functions such as, for example, but not limited to, generating and outputting a notification message to the user of the medical device indicating the presence of a USB cable connection, disabling one or more function associated with the operation of the medical device, such as, for example, disabling the display unit/output unit so no information is generated to the user of the medical device, or disabling the analyte level determination function of the medical device.

In one embodiment, the configuration of the contacts and associated electrical connection used to detect the presence or insertion of the USB cable into the USB interface, port or receptacle port of the medical device, provides for an indication of the presence of USB cable, regardless of whether the opposite end of the USB cable is connected to a powered electronic device such as a personal computer. In this manner, in one aspect, the contacts and the control unit may be configured to detect the presence or insertion of a USB cable and provide an indication to the user of such detection.

Accordingly, in one aspect of the present disclosure, there is provided a combination including detecting the presence of an electrical connection in a data port of a medical device, the presence of the electrical connection associated with a variation in a signal level resulting from the electrical connection in the data port, and generating a control signal in response to the detected presence of the electrical connection in the data port, wherein generating the control signals includes one or more of outputting a notification associated with the presence of the electrical connection in the data port or modifying one or more operational parameters associated with the medical device.

In one aspect, the modified one or more operational parameters associated with the medical device may include analysis of a fluid sample.

The fluid sample may include a blood sample.

The analysis may include determining an analyte level associated with the fluid sample.

The analyte level may include glucose level.

In another aspect, detecting the presence of an electrical connection may include monitoring a position of a contact portion relative to a ground terminal of the medical device.

The position of the contact portion may be electrically coupled to the ground terminal of the medical device when the presence of the electrical connection is detected.

The contact portion may include a plurality of contact portions.

In yet another aspect, detecting the presence of an electrical connection may include monitoring a position of a plurality of contact portions relative to each other.

The position of the plurality of contact portions may be separated by a predetermined distance and electrically separated.

The plurality of contact portions may be positioned such that at least a section of each contact portion is electrically coupled to each other.

Furthermore, when the at least the section of each contact portion is electrically coupled to each other, the electrical connection in the data port may be detected.

In another embodiment, a cable port may comprise a cable port receptacle configured to receive a cable, a cable port receptacle shield provided within the cable port receptacle, where the cable port receptacle shield is coupled to a ground, and one or more contacts configured for detection of an insertion of a cable into the cable port receptacle.

The cable port receptacle may be configured to receive a universal serial bus (USB) cable.

Moreover, the cable port receptacle may be configured to receive a cable selected from USB-A, USB-B, mini-USB-A, mini-USB-B, micro-USB-A, micro-USB-B and USB On-The-Go micro and mini USB-A or USB-B cables.

The one or more contacts may be configured to contact the cable port receptacle shield when the cable is inserted into the cable port receptacle.

The one or more contacts may comprise two contacts.

Furthermore, the two contacts may be configured to connect via a conductive portion of the cable.

The conductive portion of the cable may include a cable connection shield.

In one aspect, the two contacts may be configured to touch when the cable is inserted into the cable port receptacle.

The two contacts may not touch the cable port receptacle shield.

In one aspect, the cable port may comprise an insulating barrier positioned between the cable port receptacle shield and the two contacts.

In another aspect, the cable port may comprise an insulating barrier positioned between the two contacts and the cable.

Various other modifications and alterations in the structure and method of operation of this disclosure will be apparent to those skilled in the art without departing from the scope and spirit of the present disclosure. Although the present disclosure has been described in connection with specific embodiments, it should be understood that the present disclosure as claimed should not be unduly limited to such specific embodiments. It is intended that the following claims define the scope of the present disclosure and that structures and methods within the scope of these claims and their equivalents be covered thereby.

What is claimed is:

1. A method, comprising:
detecting the presence of an electrical connection in a data port of a medical device, the presence of the electrical connection associated with a variation in a signal level resulting from the electrical connection in the data port; and
generating a control signal in response to the detected presence of the electrical connection in the data port;
wherein generating the control signal includes one or more of outputting a notification associated with the presence of the electrical connection in the data port or modifying one or more operational parameters associated with the medical device.

2. The method of claim 1, wherein the modified one or more operational parameters associated with the medical device includes analysis of a fluid sample.

3. The method of claim 2, wherein the fluid sample includes a blood sample.

4. The method of claim 2, wherein the analysis includes determining an analyte level associated with the fluid sample.

5. The method of claim 4, wherein the analyte level includes glucose level.

6. The method of claim 1, wherein detecting the presence of an electrical connection includes monitoring a position of a contact portion relative to a ground terminal of the medical device.

7. The method of claim 6, wherein the position of the contact portion is electrically coupled to the ground terminal of the medical device when the presence of the electrical connection is detected.

8. The method of claim 6, wherein the contact portion includes a plurality of contact portions.

9. The method of claim 1, wherein detecting the presence of an electrical connection including monitoring a position of a plurality of contact portions relative to each other.

10. The method of claim 9, wherein the position of the plurality of contact portions are separated by a predetermined distance and electrically separated.

11. The method of claim 9, wherein the plurality of contact portions are positioned such that at least a section of each contact portion is electrically coupled to each other.

12. The method of claim 11, wherein when the at least the section of each contact portion is electrically coupled to each other, the electrical connection in the data port is detected.

13. A cable port, comprising:
a cable port receptacle configured to receive a cable;
a cable port receptacle shield provided within the cable port receptacle, where the cable port receptacle shield is coupled to a ground; and
one or more contacts configured for detection of an insertion of a cable into the cable port receptacle.

14. The cable port of claim 13, wherein the cable port receptacle is configured to receive a universal serial bus (USB) cable.

15. The cable port of claim 14, wherein the cable port receptacle is configured to receive a cable selected from USB-A, USB-B, mini-USB-A, mini-USB-B, micro-USB-A, micro-USB-B and USB On-The-Go micro and mini USB-A or USB-B cables.

16. The cable port of claim 13, wherein the one or more contacts are configured to contact the cable port receptacle shield when the cable is inserted into the cable port receptacle.

17. The cable port of claim 13, wherein the one or more contacts comprises two contacts.

18. The cable port of claim 17, wherein the two contacts are configured to connect via a conductive portion of the cable.

19. The cable port of claim 18, wherein the conductive portion of the cable includes a cable connection shield.

20. The cable port of claim 17, wherein the two contacts are configured to touch when the cable is inserted into the cable port receptacle.

21. The cable port of claim 20, wherein the two contacts do not touch the cable port receptacle shield.

22. The cable port of claim 21, further comprising an insulating barrier positioned between the cable port receptacle shield and the two contacts.

23. The cable port of claim 20, further comprising an insulating barrier positioned between the two contacts and the cable.
